(19) United States
(12) Patent Application Publication
(10) Pub. No.: US 2021/0077314 A1
(43) Pub. Date: Mar. 18, 2021

(54) Sanitary Pad

(71) Applicant: Mbali Ncube, Stamford, CT (US)

(72) Inventor: Mbali Ncube, Stamford, CT (US)

(21) Appl. No.: 16/574,561

(22) Filed: Sep. 18, 2019

(51) Int. Cl. A61F 13/476; A61F 13/56; A61F 13/475

(52) U.S. Cl. CPC A61F 13/476; A61F 13/475; A61F 13/5616

(57) ABSTRACT

A sanitary pad, including an elongated body having an arcuate anterior end and a pointed posterior end. A first absorbent region of the elongate body is adapted, when the sanitary pad is used, to extend over the perineal region of a user's body, and a second region of the elongate body is adapted, when the sanitary pad is used, to extend over the anal region of a user's body. A pair of wings, adapted, when the sanitary pad is in use, to wrap around the user's underpants, extend perpendicularly outwardly from a midpoint of the elongated body.

### BACKGROUND OF THE DISCLOSED TECHNOLOGY

Sanitary pads, used to externally absorb menstrual discharge, have been known since 1888. Such pads are adapted to be placed exteriorly against the skin around the genital openings, so as to absorb menstrual discharge. However, because these pads are external, and can move around relative to the user's body, many women experience leakages while using pads, for example due to pad saturation, or to movement of the pad relative to the body.

Leakage problems are particularly prevalent when women are asleep, and are not aware of shifting of the pad relative to their bodies, or of saturation of the pad.

Many pad structure have been proposed in an attempt to prevent leakage from pads during the night, some of which are mentioned hereinbelow. However, to date, the problem has not been solved.

U.S. Patent Application Publication No. 2013/0053808 to Hill discloses a sanitary pad including a forward semi-square raise absorbent portion, a narrow center raise that becomes concave and extends substantially over the forward portion of the female anatomy and the forward portion of the labia, a rearward raise peaked absorbent portion that extends from a rear gradual raise and begins in the pudendal cleft rearward portion of the labia and extends to the perineum and slightly ending at the tip before entering between the buttocks, and center flaps which help absorb leakage.

U.S. Pat. No. 7,857,799 to Lavash et al. shows a feminine hygiene device providing for improved static and dynamic body fit. The feminine hygiene device includes a topsheet, a backsheet joined to the topsheet, and an absorbent core disposed between the topsheet and the backsheet. The feminine hygiene device has a longitudinal axis FL. The absorbent core has a core longitudinal axis L substantially coinciding with the longitudinal axis FL of the feminine hygiene device. Feminine hygiene device 20 also includes side extensions, commonly referred to as “wings,” designed to wrap around the sides of the pudendal region of the panties of the user of feminine hygiene device.

U.S. Pat. No. 9,339,423 to Hashino et al. shows an absorbent article including a liquid-permeable top sheet provided on the skin side, a liquid-impermeable back sheet provided on the clothing side, and a liquid-retaining absorbent body situated between the top sheet and the back sheet. The absorbent article further has a body section and a pair of wing sections extending from the body section in the widthwise direction.

U.S. Patent Application Publication No. 2006/0184150 to Noel shows a catamenial device, that can be a sanitary napkin or pantiliner, having a body-contacting surface and a topsheet at least a portion of which has a plurality of apertures. The device has a liquid impervious backsheet joined to the topsheet, and an absorbent core disposed between the topsheet and backsheet. The sanitary napkin has a longitudinal axis and may also be provided with additional features commonly found in sanitary napkins, including “wings” or “flaps” and/or a fluid acquisition layer between the topsheet and the absorbent core to promote fluid transport from the topsheet to the absorbent core.

U.S. Patent Application Publication No. 2010/0305541 to Noda et al. discloses a sanitary napkin which consists of a strip-shaped absorbent article main body including a liquid-retaining absorber, a topsheet of liquid-permeable covering a surface of the absorber, and a backsheet of liquid-nonpermeable supporting the absorber from the back. On both sides of the main body, right and left wings are formed to assist fixing of the sanitary napkin 11 to underpants. On a back surface of the main body, a strip-shaped support means including a belt-shaped member and an attaching sheet is provided for supporting the main body when the sanitary napkin is used. The strip-shaped support means can be pulled out backward in the longitude direction of the main body.

There is thus a need in the art for a sanitary pad that is sufficiently absorbent, and suitably structured, so as to prevent leakage from the pad, particularly during hours in which the user is asleep.

### SUMMARY OF THE DISCLOSED TECHNOLOGY

The disclosed technology described herein addresses a need, unfulfilled in the prior art, for providing a sanitary pad which is structured and configured to prevent leakage of the pad, even when a region thereof directly adjacent the genital openings is saturated.

It is an objective of the disclosed technology to prevent blood flow to the intergluteal cleft or region, or any rearward flow of blood or other fluid toward the anal region of the user.

It is another objective of the disclosed technology to reduce or prevent leakage of fluid into the intergluteal region or cleft and/or any upward fluid flow, by absorbing excess fluid within the sanitary pad.

It is a further objective of the disclosed technology to enable the user to move freely during their menstrual period, without fear of blood leaking onto their clothes, in both an upright position and a supine position.

In accordance with one aspect of an embodiment of the disclosed technology, there is provided a sanitary pad, including an elongated body having an arcuate anterior end and a pointed posterior end. A first absorbent region of the elongate body is adapted, when the sanitary pad is used, to extend over the perineal region of a user's body, and a second region of the elongate body is adapted, when the sanitary pad is used, to extend over the anal region of a user's body. A pair of wings, adapted, when the sanitary pad is in use, to wrap around the user's underpants, extend perpendicularly outwardly from a midpoint of the elongated body.

In some embodiments, the first absorbent region includes more absorbent material than the second absorbent region.

In some embodiments, the elongated body tapers in width from approximately the midpoint to an apex of the pointed posterior end. As such, the elongated body fits comfortably, when the sanitary pad is in use, within a user's intergluteal region. In some embodiments, the elongated body has a concave shape, and includes an upward arch on the pointed posterior end. As such, the concave shape of the elongated body corresponds to the anatomy or shape of the perineal region of a user.

In some embodiments, the sanitary pad further includes a channeling mechanisms adapted to channel excess fluid, not absorbed by the first absorbent region, from the first absorbent region to the second absorbent region.

In some embodiments, a central portion of the second absorbent region has a greater number of layers of absorbent material than lateral edge portions of the second absorbent region.

In some embodiments, a majority of the first absorbent region is anterior to the midpoint. In some cases, the entire first absorbent region is anterior to the midpoint. In some embodiments, longitudinal majority of the second absorbent region is posterior to the midpoint.

In some embodiments, the first absorbent region has a first longitudinal length, the second absorbent region has a second longitudinal length, and the second longitudinal length is greater than the first longitudinal length.

In accordance with one aspect of another embodiment of the disclosed technology, there is provided a method of using a sanitary pad by a user, which includes placing the sanitary pad described hereinabove on a crotch panel of underpants, such that an adhesive surface of the sanitary pad engages an interior surface of the crotch panel, and a liquid permeable surface of the sanitary pad is exposed. The method further includes engaging an adhesive surface of the wings of the sanitary pad to an exterior surface of the crotch panel. The user then wears the underpants such that the first absorbent region extends over the perineal region of the user's body and the second absorbent region extends over the anal region of the user's body.

In accordance with one aspect of yet another embodiment of the disclosed technology, there is provided a sanitary pad, including an elongated body having an arcuate anterior end and a pointed posterior end, and first and second absorbent regions. The elongated body tapers in width from a midpoint thereof to an apex of the pointed posterior end so as to fit comfortably, when the sanitary pad is in use, within a user's intergluteal region. The elongated body has a concave shape, and includes an upward arch on the pointed posterior end such that the concave shape corresponds to the anatomy or shape of the perineal region of a user.

In some embodiments, the first absorbent region includes more absorbent material than the second absorbent region. In some embodiments, when the sanitary pad is in use, the first absorbent region is adapted to extend over the perineal region of a user's body and the second absorbent region is adapted to extend over the anal region of a user's body.

In some embodiments, the sanitary pad further includes a channeling mechanism adapted to channel excess fluid, not absorbed by the first absorbent region, from the first absorbent region to the second absorbent region.

In some embodiments, a central portion of the second absorbent region has a greater number of layers of absorbent material than lateral edge portions of the second absorbent region.

In some embodiments, a majority of the first absorbent region is anterior to the midpoint. In some embodiments, a longitudinal majority of the second absorbent region is posterior to the midpoint.

In some embodiments, the first absorbent region has a first longitudinal length, the second absorbent region has a second longitudinal length, and the second longitudinal length is greater than the first longitudinal length.

Any device or step to a method described in this disclosure can comprise, or consist of, that which it is a part of, or the parts which make up the device or step. The term “and/or” is inclusive of the items which it joins linguistically, and each item by itself. Any device can be “substantially” as described, if specified in the claims, “substantially” being defined as “within a tolerance level know in the art” or “within 95% thereof”.

### BRIEF DESCRIPTION OF THE DRAWINGS

FIG. 1 shows a perspective view of a sanitary pad according to an embodiment of the disclosed technology.

FIG. 2 shows a planar top view illustration of the sanitary pad of FIG. 1.

FIG. 3 shows a perspective view demonstrating the curvature of the sanitary pad of FIGS. 1 and 2.

FIG. 4 is a schematic illustration showing the sanitary pad of FIGS. 1 to 3, in use.

### DETAILED DESCRIPTION OF EMBODIMENTS OF THE DISCLOSED TECHNOLOGY

The presently disclosed technology is directed towards a sanitary pad, having a geometry adapted to suit the female anatomy and having two absorbent regions, which prevent leakage from of menstrual discharge, particularly during hours in which the user is sleeping.

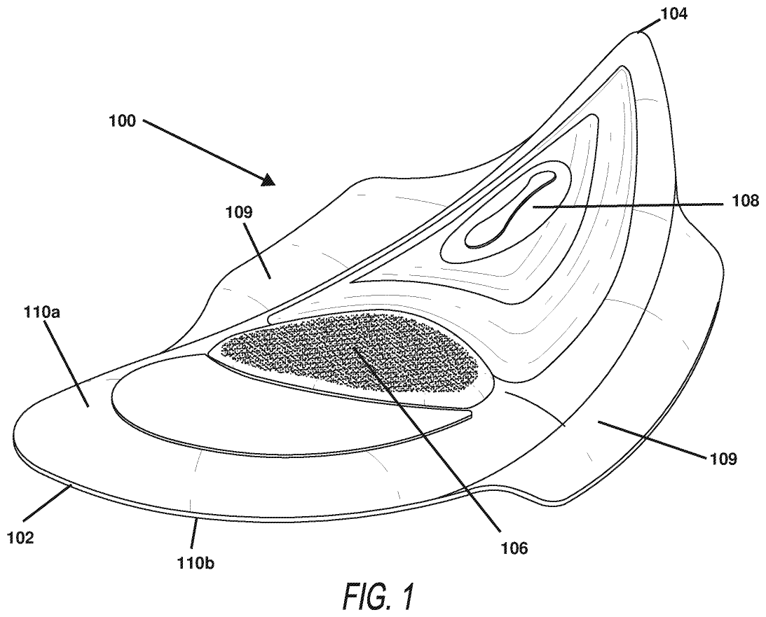

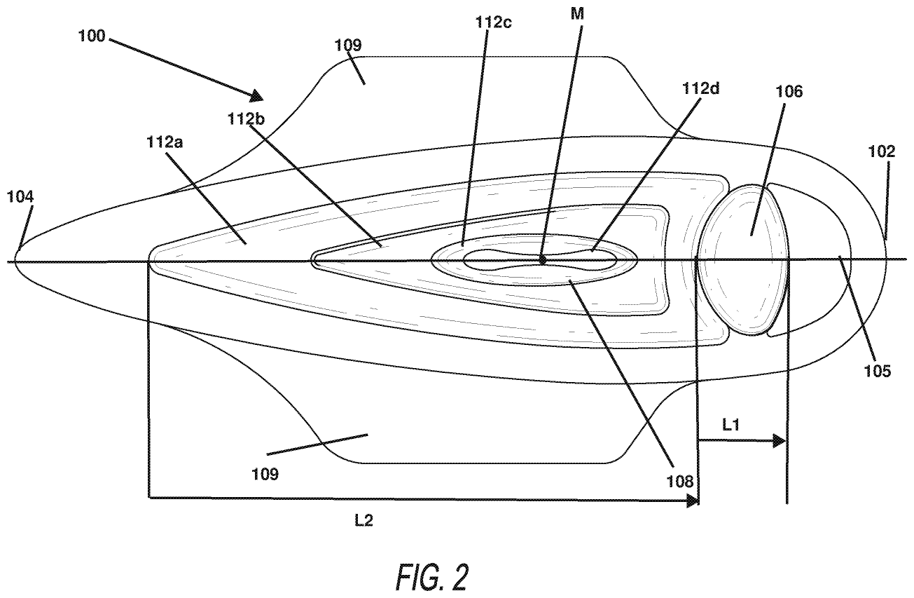

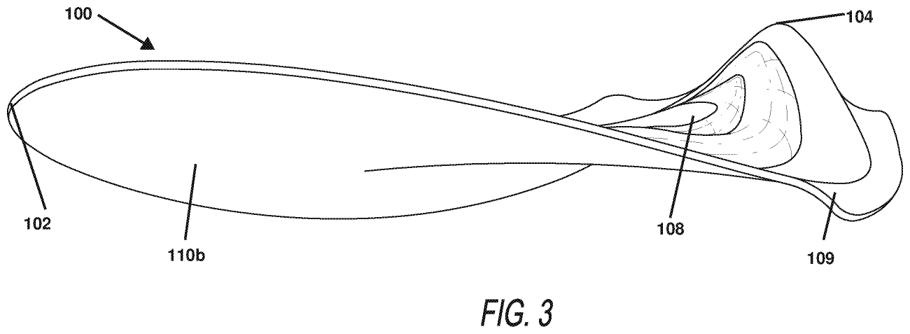

FIG. 1 shows a perspective view of a sanitary pad 100 according to an embodiment of the disclosed technology, FIG. 2 shows a planar top view illustration of the sanitary pad 100, and FIG. 3 shows a perspective view demonstrating the curvature of the sanitary pad 100.

As seen, sanitary pad 100 includes an elongated body having an arcuate anterior end 102 and a pointed posterior end 104. The sanitary pad 100 is arranged along a longitudinal axis 105 (shown clearly in FIG. 2) having a midpoint M. The elongated body further includes a first absorbent region 106 and a second absorbent region 108. A pair of wings 109 extend perpendicularly outwardly from a mid-region of the elongated body, the mid-region surrounding midpoint M of the elongated body.

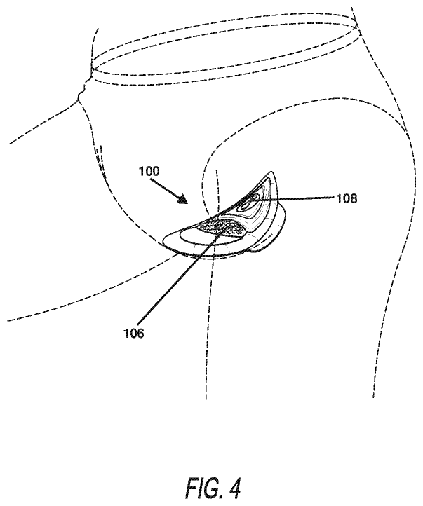

Turning to FIG. 4, it is seen that when the sanitary pad is disposed within underpants of a user along a crotch panel thereof, the first absorbent region 106 is adapted to extend over the perineal region of the user's body, and the second absorbent region 108 is adapted to extend over the anal region of the user's body. The wings 109 are folded over edges of the underpants of the user, to hold the sanitary pad in place.

The elongated body of sanitary pad 100 includes a liquid permeable upper surface 110a, adapted to engage the skin of the user, and a liquid impermeable lower surface 110b adapted to engage a garment of clothing, as seen in FIG. 4. Absorbent material is disposed between upper surface 110a and lower surface 110b, in the first and second absorbent regions 106 and 108. In some embodiments, first absorbent region 106 includes more absorbent material than second absorbent region 108.

A channeling mechanism is adapted to channel excess fluid, not absorbed by first absorbent region 106, for example due to saturation thereof, from first absorbent region 106 to second absorbent region 108. In some embodiments, the channeling mechanism includes direct contact between first and second absorbent regions 106 and 108, or between absorbent materials thereof. In other embodiments, the channeling mechanism may include a channel or other conduit forming fluid communication between the first and second absorbent regions.

As seen clearly in FIG. 2, second absorbent region 108 includes multiple layers of absorbent material, which gradually increase in dimensions. For example, a base layer 112a is greater in size than a subsequent layer 112b, which in turn is greater in size than a layer 112c, and so forth, up to a central layer 112d. As such, a central portion or area of second absorbent region 108 has a greater number of layers of absorbent material than lateral edge portions or areas of the second absorbent region.

First absorbent region 106 has a first longitudinal length, indicated in FIG. 2 by L1, and second absorbent region 108 has a second longitudinal length, indicated in FIG. 2 by L2. In some embodiments, the second longitudinal length L2 is greater than the first longitudinal length L1. In some embodiments, the second longitudinal length L2 is at least twice the length L 1 (L2≥2*L1), at least three times the length L1 (L2≥3*L1), or at least four times the length L1 (L2≥4*L1).

As seen clearly in FIG. 2, the elongated body of sanitary pad 100 tapers in width, from approximately midpoint M of longitudinal axis 105, to an apex of pointed posterior end 104. This structure enables the sanitary pad 100 to fit comfortably within a user's intergluteal region when the pad is in use, as shown in FIG. 4.

In some embodiments, a majority of first absorbent region 106, or the entirety thereof, is anterior to midpoint M, as shown in FIG. 2. In some embodiments, a longitudinal majority of second absorbent region 108, or a majority of length L2, is posterior to midpoint M.

As seen clearly in FIGS. 1 and 3, sanitary pad 100, and specifically the elongated body thereof, has a concave shape, and includes an upward arch on pointed posterior end 104. This concave shape corresponds to the anatomy or shape of the perineal region of a user. It is a particular feature of the disclosed technology that the sanitary pad has this concave shape at all times, both prior to use and during use.

It is appreciated by people of skill in the art that the sanitary pad 100 is shaped and configured to fit within the intergluteal cleft cavity, between the gluteus maximus muscles, and to prevent blood or fluid flow into, and from, the intergluteal cleft. It is further appreciated that the curved shape of sanitary pad 100 is designed and configured to fit comfortably on the female genital area, thus preventing discomfort during the menstrual period.

In the context of the present application and claims, the perineal region is defined as the region extending between a woman's thighs, from the pubic arch to the transverse perineal muscles, and includes the urethra and vaginal opening.

In the context of the present application and claims, the anal region is defined as a substantially triangular anatomical region extending from the transverse perineal muscles to the coccyx, or tailbone, which includes the anus, which is the anal terminus of the digestive tract.

In the context of the present application and claims, the intergluteal region is defined as an anatomical region extending between the buttocks cheeks, or gluteal muscles, of a human.

In the context of the present application and claims, the crotch panel of underpants is defined as the portion of the underpants which, when worn, extends between a woman's thighs, and covers the urethra, the vaginal opening, and the anus.

“Substantially” and “substantially shown,” for purposes of this specification, are defined as “at least 95%,” or as otherwise indicated. Any device may “comprise” or “consist of” the devices mentioned there-in, as limited by the claims.

It should be understood that the use of “and/or” is defined inclusively such that the term “a and/or b” should be read to include the sets: “a and b,” “a or b,” “a,” “b.”

While the disclosed technology has been taught with specific reference to the above embodiments, a person having ordinary skill in the art will recognize that changes can be made in form and detail without departing from the spirit and the scope of the disclosed technology. The described embodiments are to be considered in all respects only as illustrative and not restrictive. All changes that come within the meaning and range of equivalency of the claims are to be embraced within their scope. Combinations of any of the methods, systems, and devices described herein above are also contemplated and within the scope of the invention.

I claim:

1. A sanitary pad, comprising:
an elongated body having an arcuate anterior end and a pointed posterior end;
a first absorbent region adapted, when said sanitary pad is used, to extend over the perineal region of a user's body;
a second absorbent region adapted, when said sanitary pad is used, to extend over the anal region of a user's body; and
a pair of wings extending perpendicularly outwardly from a midpoint of the elongated body.

2. The sanitary pad of claim 1, wherein said first absorbent region includes more absorbent material than said second absorbent region.

3. The sanitary pad of claim 1, wherein said elongated body tapers in width from approximately said midpoint to an apex, of said pointed posterior end so as to fit comfortably, when said sanitary pad is in use, within a user's intergluteal region.

4. The sanitary pad of claim 1, wherein said elongated body has a concave shape, and includes an upward arch on said pointed posterior end such that said concave shape corresponds to the anatomy or shape of the perineal region of a user.

5. The sanitary pad of claim 1, further comprising a channeling mechanism adapted to channel excess fluid, not absorbed by said first absorbent region, from said first absorbent region to said second absorbent region.

6. The sanitary pad of claim 1, wherein a central portion of said second absorbent region has a greater number of layers of absorbent material than lateral edge portions of said second absorbent region.

7. The sanitary pad of claim 1, wherein a majority of said first absorbent region is anterior to said midpoint.

8. The sanitary pad of claim 7, wherein the entirety of said first absorbent region is anterior to said midpoint.

9. The sanitary pad of claim 1, wherein a longitudinal majority of said second absorbent region is posterior to said midpoint.

10. The sanitary pad of claim 1, wherein said first absorbent region has a first longitudinal length, said second absorbent region has a second longitudinal length, and said second longitudinal length is greater than said first longitudinal length.

11. A method of using a sanitary pad by a user, comprising:
placing the sanitary pad of claim 1 on a crotch panel of underpants, such that an adhesive surface of the sanitary pad engages an interior surface of said crotch panel, and a liquid permeable surface of the sanitary pad is exposed;
engaging an adhesive surface of the wings of said sanitary pad to an exterior surface of said crotch panel; and
wearing said underpants such that said first absorbent region extends over the perineal region of the user's body and the second absorbent region extends over the anal region of the user's body.

12. A sanitary pad, comprising:
an elongated body having an arcuate anterior end and a pointed posterior end; and
first and second absorbent regions,
wherein said elongated body tapers in width from a midpoint thereof to an apex of said pointed posterior end so as to fit comfortably, when said sanitary pad is in use, within a user's intergluteal region, and
wherein said elongated body has a concave shape, and includes an upward arch on said pointed posterior end such that said concave shape corresponds to the anatomy or shape of the perineal region of a user.

13. The sanitary pad of claim 12, wherein said first absorbent region includes more absorbent material than said second absorbent region.

14. The sanitary pad of claim 13, wherein, when said sanitary pad is in use, said first absorbent region is adapted to extend over the perineal region of a user's body and the second absorbent region is adapted to extend over the anal region of a user's body.

15. The sanitary pad of claim 12, further comprising a channeling mechanism adapted to channel excess fluid, not absorbed by said first absorbent region, from said first absorbent region to said second absorbent region.

16. The sanitary pad of claim 12, wherein a central portion of said second absorbent region has a greater number of layers of absorbent material than lateral edge portions of said second absorbent region.

17. The sanitary pad of claim 12, wherein a majority of said first absorbent region is anterior to said midpoint.

18. The sanitary pad of claim 12, wherein a longitudinal majority of said second absorbent region is posterior to said midpoint.

19. The sanitary pad of claim 12, wherein said first absorbent region has a first longitudinal length, said second absorbent region has a second longitudinal length, and said second longitudinal length is greater than said first longitudinal length.
